(19) United States
(12) Patent Application Publication
(10) Pub. No.: US 2016/0320415 A1
(43) Pub. Date: Nov. 3, 2016

(54) FAST RECONSTITUTING REAGENT PELLETS

(71) Applicant: Victor Manneh, Encinitas, CA (US)

(72) Inventor: Victor Manneh, Encinitas, CA (US)

(73) Assignee: XEN BIOFLUIDX, INC., San Diego, CA (US)

(21) Appl. No.: 14/106,930

(22) Filed: Dec. 16, 2013

(51) Int. Cl. G01N 33/86; B05D 1/02

(52) U.S. Cl. CPC G01N 33/86; B05D 1/02

(57) ABSTRACT

Dry, pelletized reagents are described which rapidly reconstitute into active reagent solutions when contacted with an appropriate liquid sample or other appropriate solution, including heterogeneous reagent pellets made from two or more separate reagent solutions in such manner that components in the separate reagent solutions which will normally react with each other in solution do not substantially react. Also described are reagent dispenser packages and assay devices containing dry pelletized reagents.

### RELATED APPLICATIONS

Not Applicable.

### FIELD OF THE INVENTION

The present invention relates to the preparation of dry reagents, including for assay reagents.

### BACKGROUND OF THE INVENTION

The following discussion is provided solely to assist the understanding of the reader, and does not constitute an admission that any of the information discussed or references cited constitute prior art to the present invention.

Typically in biological and biochemical assays, reagents are immersed in a liquid, most commonly an aqueous medium. However, it may be advantageous in many cases to have one or more reagents in dry form until the assay is actually performed. Dry reagents have been prepared in a variety of ways, including drying with heat, under vacuum, and using lyophilization.

For example, Hawkins et al., U.S. Pat. No. 5,625,036 describes a prothrombin time (PT) reagent preparation containing recombinant human tissue factor, phospholipids, and other components dispensed into vials and dried using lyophilization.

Brown, U.S. Pat. No. 5,314,695, describes a dried PT reagent containing native or recombinant human tissue factor embedded in the lipid bilayer of defined liposomes. The liposome solution may be used in liquid form or lyophilized for storage and reconstituted for use.

Lee, U.S. Pat. No. 6,733,985, describes dry reagents for a prothrombin time test for monitoring extrinsic blood coagulation activities, and preparation of such dry reagents by air-drying at temperatures above 10 degrees C.

Calatizis et al., US Pat Appl Publ 2010/0190193, published Jul. 29, 2010, describes a dried diagnostic composition for use in a viscoelastic analysis of a test liquid. The composition contains at least one activator of coagulation, such as tissue factor, celite, ellagic acid, sulfatit, kaolin, silica, RNA, and mixtures of those activators. The composition may also contain a calcium salt and/or one or more inhibitors or other coagulation components or factor. The composition is dried, such as by lyophilization, preferably in the container in which the composition will be used. The composition and container may be used with a diagnostic device such as the device described in U.S. Pat. No. 5,777,215.

In some cases, dried reagents have been prepared in bead or pellet form. Gombotz et al., U.S. Pat. No. 6,569,458 describes a process for preparing small particles of biologically active molecules, which involves spraying a solution of the molecules into a very cold liquefied gas, and lyophilizing the frozen droplets to produce dry spheres. Ultrasonic energy or homogenization may be used to reduce the size of the particles to diameters of 0.1 to 10 micrometers.

Buhl et al., U.S. Pat. No. 6,251,684, describes reagent beads in the size range from a minimum of 1.5 mm up to 10 mm for pharmaceutical preparations of analytical procedures. The beads are said to dissolve quickly, typically in less than 30, 10, 1, or 0.3 seconds.

### SUMMARY OF THE INVENTION

The present invention concerns fast reconstituting and/or heterogeneous reagent pellets, as well as dispensers containing the same. Fast reconstituting reagent pellets are particularly useful in assays and other applications in which reagents must be quickly available and/or available substantially completely at one time. Particular applications include elastometry and other hemostasis assays, other blood assays, and nucleic acid amplification assay, among others. In the addition, the invention concerns the manufacture and use of heterogeneous pellets, which may be but are not necessarily fast reconstituting pellets. Such heterogeneous pellets contain components which would react with each other (e.g., enzymatic reaction or binding reaction) under normal conditions, but which are combined in a pellet in a manner which does not allow sufficient reaction to create a difficulty (e.g., does not consume a substantial amount of enzyme substrate). The invention also concerns liquid reagent reconstitution devices which utilize liquid transfer or dispensing devices containing dry reagents, such as the dry, pelletized reagents as described.

Thus, a first aspect of the invention concerns a liquid reagent reconstitution device which includes a liquid dispenser (e.g., a pipette tip or other transfer device) within which is packaged one or more dry reagents, e.g., 1, 2, 3, 4, 5, 6, or more dry regents. In certain cases, the dry reagents are in the form of dry reagent pellets (also referred to as pelletized reagent), which can be rapidly reconstitutable reagent pellets. In some cases, the device is a single-use device.

In certain embodiments, the liquid dispenser is a liquid dispensing device (e.g., a pipette tip); which may be a disposable or single-use device; at least 2, 3, 4, 5, 6, or more pellets are packaged within the liquid dispenser (such as a pipette tip); in embodiments in which a plurality of pellets are packaged within a disposable liquid dispenser (e.g., pipette tip), the pellets are of 1, 2, 3, 4, or more different types (usually containing different reagents); pellets of different types (e.g., different types containing different reagents) are different colors; the dispensing device contains a dessicant, e.g., impregnated in or coated on a retaining barrier, e.g., a porous barrier.

In certain embodiments, the reagents in the dry reagent pellets are selected and adapted for a pre-selected assay, e.g., a blood assay, an enzymatic assay, a binding assay, a nucleic acid amplification assay; use of the pelletized reagent in the pre-selected assay produces substantially equivalent results as results produced using liquid reagents.

As indicated above, in some cases, at least one dry pelletized reagent is packaged within a pipette tip; the pipette time may includes a pellet retainer distal to the dispensing orifice of the pipette tip; the pipette tip also includes a desiccant, e.g., impregnated in or coated on or within the pellet retainer; the pellet retain is situated distal to the point where liquid would reach during normal use of the pipette tip.

In certain embodiments, the reagent pellet(s) comprise a dry, pelletized reagent composition selected and adapted for use in performing a hemostasis measurement assay. In certain embodiments, the hemostasis measurement is part of a blood coagulation assay, a blood clotting assay, a platelet function assay, a thromboelastomery, or a fibrinolytic assay; the pellet contains at least one reagent selected from the group consisting of an enzyme, an enzyme substrate, a mineral salt, an enzyme activator, a coagulation activator, and a fibrinolysis inhibitor; the pellet contains at least one reagent selected from the group consisting of Tissue Factor (thromboplastin) which may be recombinant Tissue Factor, polybrene, kaolin, silica, CaCl2, and heparinase; the pelletized reagent composition contains a plurality of different types of pellets, each containing different reagents (i.e., made from different reagent solutions) in which the different types of pellets may be, are not necessarily, different colors; use the composition in the assay produces substantially equivalent results as results produced using liquid reagents, e.g., in a hemostasis assay such as a thromboelastometry assay.

Also in certain embodiments, the dry reagent includes at least one reagent pellet (which may be a fast reconstituting reagent pellet); the reagent pellet(s) include a dry, pelletized reagent composition selected and adapted for cellular aggregation, e.g., blood cell aggregation, such as aggregation of red blood cells; the reagent pellet(s) include at least one cellular aggregation agent, e.g., one or more antibodies (which may include intact antibodies, antibody fragments, single chain antibodies, and the like) and will commonly be anti-cell surface antibodies, and/or one or more lectins (e.g., lectins which bind cell surface oligosaccharides). Other examples of cell aggregating agents which may be incorporated include phytohaemagglutinin (PHA, including PHA-E and PHA-L), Soy Bean Agglutinin (SBA), Concanavalin A (ConA), lens culinaris agglutinin (LCA), Rininus comunic agglutinin (RCA), Viscum album agglutinin (VAA), wheat germ agglutinin (WGA), collagen, fibrinogen, and von Willebrand Factor (vWF).

In beneficial embodiments, the pelletized reagent includes at least one dry reagent pellet which dissolves in 3 seconds or less in use in the assay or other intended procedure without forced fluid flow (e.g., via centrifugation). In advantageous embodiments, the pellet dissolves in 2, 1.5, 1, 0.5, 0.4, 0.3, 0.2, or 0.1 seconds or less.

In particular embodiments, the pellet has a diameter of 5, 4, 3, 2, or 1 mm or less, or 700, 500, 400, 300, 200, 100, 70, 50, 40, 30, 20, 10, or 5 micrometer or less, or is in a range of 5 to 20 micrometer, 5 to 50 micrometer, 10 to 50 micrometer, 10 to 100 micrometer, 20 to 50 micrometer, 20 to 100 micrometer, 50 to 100 micrometer, 50 to 200 micrometer, 50 to 500 micrometer, 100 to 500 micrometer, 100 micrometer to 1 mm, 200 to 500 micrometer, 200 to 700 micrometer, 200 micrometer to 1 mm, 200 micrometer to 2 mm, 0.5 mm to 0.7 mm, 0.5 mm to 1 mm, 0.5 mm to 2 mm, 0.5 mm to 5 mm, 0.7 mm to 1 mm, 0.7 mm to 2 mm, 0.7 mm to 5 mm, 1 mm to 2 mm, 1 mm to 3 mm, 1 mm to 4 mm, 1 mm to 5 mm, 2 mm to 3 mm, 2 mm to 4 mm, or 2 mm to 5 mm, or other size range defined by taking any two of the specified values as inclusive endpoints of a range.

A related aspect of the invention provides a fast reconstituting, dry, pelletized reagent composition selected for use in a reaction, e.g., an in vitro biological reaction, where the reagent pellets dissolve within 2, 1.5, 1, 0.5, 0.4, 0.3, 0.2, or 0.1 seconds or less in use for the reaction.

In particular embodiments, the biological reaction is an assay reaction (a reaction carried out as part of a biological or biochemical assay), a sample preparation reaction (such as a blood cell aggregation reaction, sample preservation, enzyme inactivation, pH adjustment, or target cleavage, .e.g, protein or nucleic acid cleavage); the pelletized reagent composition is packaged as a single use reagent aliquot (for example, for any of the applications mentioned for the reagent pellets); use of the composition in a pre-selected assay produces substantially equivalent results as results produced using liquid reagents.

Likewise, in particular embodiments, the pellets are as specified for the preceding aspect or an embodiment thereof which are consistent with this aspect.

Thus, in particular embodiments, the pellet has a diameter of 5, 4, 3, 2, or 1 mm or less, or 700, 500, 400, 300, 200, 100, 70, 50, 40, 30, 20, 10, or 5 micrometer or less, or is in a range of 5 to 20 micrometer, 5 to 50 micrometer, 10 to 50 micrometer, 10 to 100 micrometer, 20 to 50 micrometer, 20 to 100 micrometer, 50 to 100 micrometer, 50 to 200 micrometer, 50 to 500 micrometer, 100 to 500 micrometer, 100 micrometer to 1 mm, 200 to 500 micrometer, 200 to 700 micrometer, 200 micrometer to 1 mm, 200 micrometer to 2 mm, 0.5 mm to 0.7 mm, 0.5 mm to 1 mm, 0.5 mm to 2 mm, 0.5 mm to 5 mm, 0.7 mm to 1 mm, 0.7 mm to 2 mm, 0.7 mm to 5 mm, 1 mm to 2 mm, 1 mm to 3 mm, 1 mm to 4 mm, 1 mm to 5 mm, 2 mm to 3 mm, 2 mm to 4 mm, or 2 mm to 5 mm, or other size range defined by taking any two of the specified values as inclusive endpoints of a range.

Likewise, in certain embodiments, the reagent pellets are selected and adapted for use in performing a hemostasis measurement assay, e.g., as part of a blood coagulation assay, a blood clotting assay, a platelet function assay, or a fibrinolytic assay; the assay is a thromboelastometry assay; the dry reagent pellet include at least one of an enzyme, an enzyme substrate, a mineral salt, an enzyme activator, a coagulation activator, and a fibrinolysis inhibitor; the pellet includes at least one of Tissue Factor (thromboplastin), polybrene, kaolin, silica, CaCl2, and heparinase.

Likewise, in some embodiments, the reagent pellet includes at least one cell aggregation agent, e.g., at least one blood cell aggregation agent; the blood cell aggregation agent includes at least one of an anti-cell surface antibody, at least one lectin binding cell surface oligosaccharides, fibrinogen, and von Willebrand Factor.

In certain embodiments, the assay is an infectious disease marker assay, a cardiac marker assay, a CNS marker assay, a diabetes marker, or a blood component assay.

In some embodiments, the composition includes a plurality of different types of pellets (e.g., 2, 3, 4, 5, or 6 different types), each including different reagents; pellets containing different reagents may be different colors.

For certain embodiments, at least one (or at least 2, 3, 4, 5, 6, or even more) dry reagent pellet is packaged within a pipette tip; a pipette tip containing at least one dry reagent pellet also includes a pellet retainer distal to the dispensing orifice of the pipette tip and/or the pipette tip includes a desiccant, such as a desiccant within the pellet retainer or in a porous barrier distal to the reagent pellets.

In particular embodiments, the reagents are selected and adapted to carry out a nucleic acid amplification reaction, such as a PCR reaction.

Another aspect of the invention concerns a method for performing an assay, which includes drawing a reagent reconstitution liquid (e.g., a liquid sample or a buffer solution) into a liquid reagent reconstitution device (e.g., a pipette tip), where the device contains at least one fast reconstituting dry reagent pellet, thereby creating a reconstituted reagent solution. The method further includes dispensing the reconstituted reagent solution into an assay receptacle, and carrying out the assay using the reconstituted reagent solution.

In certain embodiments, the liquid reagent reconstitution device is as described for an aspect above, and/or the fast reconstituting dry reagent pellets are as described for an aspect above.

In particular embodiments, the device is a disposable pipette tip; the device is a sample preparation device.

For some embodiments, at least one said pellet is a heterogeneous reagent pellet; the device contains at least two different types of reagent pellets; the device contains at least one milli-pellet; the device contains a plurality of micro-pellets.

In particular embodiments, the assay is an enzymatic assay, a nucleic acid amplification assay, a binding assay, a hemostasis assay, or a thromboelastometry assay.

A related aspect concerns a single use assay cartridge that includes a reagent container having a housing with a reagent site(s) (such as a well, chamber, or zone) containing a single use quantity of a dry, pelletized reagent composition of the preceding aspect or a dry pelletized reagent composition as otherwise described herein for the present invention, and a liquid sample deposition zone, where liquid sample deposited in the deposition zone reconstitutes the dry, pelletized reagent contained therein. The liquid sample deposition zone and the reagent site may be the same or different. The assay cartridge provides an indication of one or more parameters, e.g., hemostasis parameters, of the liquid sample.

In particular embodiments, the cartridge is adapted to perform a hemostasis assay such as a thromboelastometry assay; a rotational force measurement or the time to a change in rotational force is measured in the well; the dry, pelletized reagent composition includes a coagulation activator such as thromboplastin.

In particular embodiments, the cartridge is adapted for use of a blood, plasma, serum, urine, or saliva sample; the cartridge is adapted for determining the presence or amount of a selected analyte in the sample, the dry, pelletized reagent composition includes a detectable label, an analyte binding agent, an enzyme.

An additional related aspect concerns a method for performing a coagulation or platelet function assay, and involves contacting a single use aliquot of a dry pelletized reagent composition (e.g., in a tube, plate well, or assay cartridge well) with an assay sample containing functional coagulation components, where the dry pelletized reagent composition is a dry pelletized reagent composition as described for an aspect above or an embodiment thereof, and determining the magnitude or amount of change in a parameter corresponding to coagulation or platelet function.

In certain embodiments, the dry pelletized reagent composition is packaged within a disposable liquid dispensing or measuring structure or device, e.g., a pipette tip, and a defined volume of a reconstitution medium (e.g., liquid sample, buffer solution, or water) is taken into the structure thereby reconstituting the reagent, and the reconstituted reagent is dispensed into the container or other receptacle suitable for use in the assay. The disposable liquid dispensing or measuring structure or device and/or the reagent pellets packaged therein may be as described elsewhere herein, e.g., as specified for another aspect.

Another aspect concerns a method for preparing dry heterogeneous reagent pellets, and involves placing a plurality of small droplets of a first reagent solution in a cryogenic environment, and prior to full freezing of the first reagent solution, contacting the droplets of the first reagent solution with droplets of a second reagent solution, thereby creating un-mixed heterogeneous pellets which have a first reagent volume and an adhered second reagent volume without substantial mixing of the first and second reagent solutions. In some cases, the method also includes contacting the two-solution reagent pellets containing the first and second reagent solutions with small droplets of a third reagent solution thereby forming three-reagent solution un-mixed heterogeneous pellets in which the third reagent solution does not substantially mix with the first or second reagent solution.

In particular embodiments, the small droplets of the first reagent solution are isolated when contacted with the droplets of second reagent solution, e.g., in separate cells such as in separate holes within an isolation block, which may, for example, be made of aluminum; at least one reagent component in the first reagent solution would substantially react (e.g., by binding and/or enzymatically reacting) with at least one component in the second reagent solution when mixed at a temperature of 15 degrees C.; within the reagent solutions used to made the heterogeneous pellet, one reagent solution contains an enzyme and another reagent solution contains a substrate for that enzyme; within the reagent solutions used to made the heterogeneous pellet, one reagent solution contains an antibody which specifically binds to a component of another reagent solution; within the reagent solutions used to made the heterogeneous pellet, one reagent solution contains a component (e.g., an oligonucleotide) which is complementary to an oligonucleotide in another reagent solution; a reagent solution contains at least one coagulation activator (e.g., thromboplastin or other activator as indicated herein) or other coagulation assay reagent.

In certain embodiments, the method further involves packaging at least one pellet in a liquid dispensing or measuring structure or device, e.g., a pipette tip. The liquid dispensing or measuring structure or device may be as specified below or for another aspect herein.

In another aspect, the invention also provides dry, fast reconstituting heterogeneous reagent pellets which include (i.e., are formed from droplets of) a first reagent solution and a second reagent solution, where the first reagent solution and the second reagent solution respectively contain reagent components which will react together in mixed solution at normal use temperature and are not substantially reacted in the pellet, and the pellet reconstitutes in 3 seconds or less, or at least one of the reagent solutions reconstitutes in 3 seconds or less, in normal use solution.

In certain embodiments, the heterogeneous pellet contains at least 2, 3, or 4 different reagent solution volumes or contains 2, 3, or 4 reagent solution volumes; the heterogeneous pellet is formed from at least 2, 3, or 4 different reagent solution volumes or contains 2, 3, or 4 reagent solution volumes; the heterogeneous pellet is formed from at least 2, 3, or 4 different reagent solution volumes or contains 2, 3, or 4 reagent solution volumes, and at least 2, 3, 4, or all of the different reagent solution are mixed or 2, 3, 4, or all of the reagent solution are mixed; the heterogeneous pellet is lyophilized and the first reagent solution and second reagent solution are mixed at a temperature sufficiently low that substantial reaction does not occur and/or are frozen sufficiently rapidly that substantial reaction does not occur.

In particular embodiments, the heterogeneous pellet contains at least 2, 3, or 4 different reagent solution volumes which reconstitute at substantially the same rate; the heterogeneous pellet contains at least 2, 3, or 4 different reagent solution volumes which reconstitute at significantly different rates; the heterogeneous pellet contains a fast reconstituting reagent solution volume and a significantly slower reconstituting reagent volume (e.g., formed from a reagent solution having a substantially higher sugar concentration and/or substantially smaller pores); the heterogeneous pellet contains a fast reconstituting reagent solution volume and a delayed reconstitution reagent solution volume, e.g., a volume coated or encapsulated with a delayed release coating.

In particular embodiments, the heterogeneous pellet or a reagent solution volume within the pellet is as specified for an aspect above, e.g., with respect to size, reconstitution rate, composition.

In certain embodiments, at least one of the pellets as specified for this aspect is packaged within a disposable dispensing or measuring structure or device (e.g., a pipette tip), for example as specified further below or for another aspect herein.

Yet another aspect concerns a method for detecting or managing coagulation-related hemostasis abnormalities, which involves conducting a coagulation or fibrinolysis assay by contacting a dry, fast reconstituting reagent pellet which contains coagulation or fibrinolysis assay reagents with a sample from a patient. The result of the assay provides an indication of the coagulation-related hemostasis status of the patient.

In particular embodiments, the reagent pellet is as described for an aspect above or embodiment there of or otherwise described herein for the present invention, consistent with the composition requirements of this aspect; the assay is conducted using a single-use cartridge; the assay involves a rotation force measurement or a determination of time to a change in rotational force or a combination thereof.

Certain advantageous embodiments of the aspects above, as well as additional aspects, are provided by packaging one or more of the fast reconstituting reagent pellets within a disposable liquid dispensing or measuring structure, e.g., a disposable pipette tip.

Thus, the invention further provides a reagent package containing at least one dry, fast-reconstituting reagent pellet packaged within a disposable liquid dispensing or measuring structure, e.g., a disposable pipette tip, wherein drawing a volume of aqueous liquid into the structure (e.g., pipette tip) results in rapid reconstitution of reagent solution from said reagent pellet.

In particular embodiments, a plurality of different types of reagent pellets in packaged within the structure, e.g., 2, 3, 4, 5, 6, 7, 8, 9, or 10 different types, or at least specified number of different types, or contains 2-5, 3-7, or 5-10 different types.

In certain embodiments, the pipette tip also includes a pellet retainer distal to the dispensing orifice of the pipette tip (i.e., such that the pellets are retained between the dispensing orifice and the pellet retainer) and/or a pellet retainer proximal to the dispensing orifice (e.g., such that the pellets are retained behind the pellet retainer); the pipette tip includes both the proximal and distal pellet retainers as described; the pipette tip also includes a desiccant, e.g., retained by or included within a pellet retainer.

In many embodiments, the structure, e.g., pipette tip, is sealed within a water-proof package, e.g., a water-proof pouch, bubble pack, or blister pack.

In some advantageous embodiments, the at least one pellet within the pipette tip or other structure includes substantially all reagents needed to conduct a selected assay; the at least one pellet includes at least one reagent selected and in a quantity suitable for performing a single hemostasis assay, e.g., Tissue Factor or heparinase.

For some embodiments, the at least one dry, fast-reconstituting reagent pellet is a plurality of pellets, which may of the same or different types, e.g., a plurality of different types of pellets containing different reagents; the at least one dry, fast-reconstituting reagent pellet includes at least one milli-pellet, e.g., 1, 2, 3, 4, 5, 6, or more milli-pellets, or at least the specified number of milli-pellets; the at least one dry, fast-reconstituting reagent pellet includes at least one micro-pellet, e.g., at least 2, 3, 5, 10, 15, 20, 25, 30, 40, 50, 60, 70, 80, 90, or 100 micro-pellets; the at least ne dry, fast-reconstituting reagent pellet includes at least one milli-pellet and at least one micro-pellet (or any combination numbers of milli-pellets and micro-pellets as just specified).

Additional embodiments will be apparent from the Detailed Description and from the claims.

### BRIEF DESCRIPTION OF THE DRAWINGS

FIG. 1 schematically shows a plurality of dry reagent pellets in a pipette tip.

### DETAILED DESCRIPTION OF THE PREFERRED EMBODIMENTS

As described in the Summary, this invention is concerned with the use of dry reagents in assays and other applications, where the reagents have been prepared in a highly advantageous, rapid reconstitution form and/or as heterogeneous pellets. That is, for fast reconstitution pellets, the reagents are prepared in a manner resulting in discrete pellets which very rapidly dissolve when contacted with an appropriate solution, usually an aqueous solution. To accomplish this, solution composition, droplet formation, droplet solidification, and solid droplet drying must be properly selected to provide sufficient dry pellet strength, reagent stability, and rapid pellet reconstitution. These properties are highly important in conducting many assays, especially ones in which the timing of reagent availability is a critical factor. Examples of such assays include blood clotting and other hemostasis assays, among many others. The invention also concerns liquid sample reconstitution devices which may contain reagent pellets as described.

For many types of assays, optimal performance is achieved using reagents already dissolved in liquid medium. However, using such liquid reagent solutions is frequently problematic due to reagent stability, handling, and assay format issues. As a result, it can be advantageous to use dried reagent preparations, e.g., lyophilized preparations. However, it is very desirable to utilize dried reagent preparations without substantial degradation of assay performance. Thus, in some cases, the present reagent pellets are prepared such that assay results using the dried pellets is substantially equivalent to assay performance using otherwise equivalent liquid reagents, for example, in a thromboelastometry or other hemostasis assay.

The present pellets may be prepared covering a broad range of sizes, typically with some differences in the processes for significantly different particle sizes. Thus, description is provided below for three general size ranges, but it should be recognized that the boundaries between size ranges are not discrete. As a result, formulations, equipment, and processes used for different size ranges can overlap. It should also be recognized that some of the formulations components, equipment, and processes will be in common between different size ranges.

A. Preparation of Fast Reconstituting Pellets with Active Components

In carrying out the preparation of either single solution or, for many applications, heterogeneous pellets for this invention, it is important for the resulting pellets to be fast reconstituting, but also for the reagent components to be immediately or at least rapidly active when the pellets are reconstituted. An important parameter for fast reconstitution is the physical structure of the dried pellet. That is, a porous structure allows fluid to penetrate very quickly to the interior of the pellet, resulting in very rapid and nearly simultaneous reconstitution of the entire pellet.

However, rapid reconstitution is insufficient to provide effective reagent pellets if some of the reagent components are inactive or poorly active following reconstitution. Proteins, which commonly are highly dependent on proper secondary, tertiary, and even quaternary structure for activity, are especially a concern in this regard. In many cases, especially for cases in which only a single polypeptide is needed for activity, tertiary structure resulting from proper protein folding is the primary limitation to maintaining protein activity following processing. As a result, following preparation of the present pellets, the protein molecules should be maintained in a conformational state such that they are immediately or at least very rapidly active following reconstitution. If the protein folding is excessively disturbed, frequently the re-folding process is slow and sometimes proper re-folding will not occur.

It was discovered that both advantageous pellet structure for fast reconstitution and maintenance of active proteins and other components can be achieved using certain types of solutions together with lyophilization conditions suitable for those solutions. In particular, conditions have been found effective for reagents useful in thromboelastometry and other hemostasis assays.

For pellets containing Tissue Factor (thromboplastin) and similar proteins, it has been found advantageous to utilize solutions containing about 10% trehalose and about 1.5 to 4% mannitol and/or sucrose. This combination has been found to allow substantially full recovery of enzymatic activity after reconstitution. It is expected this is due to substantial preservation of the native structure of the protein during freezing and lyophilization. The Tissue Factor and sugar containing solution is frozen by dropping solution droplets on liquid nitrogen.

In an advantageous lyophilization procedure, the frozen product was initially placed on a pre-cooled −40 degree C. lyophilizer shelf. The chamber pressure was reduced slightly to 600 mTorr and the shelf temperature was increased to −30 degrees C., held at that temperature for 2 hours and reduced to −40 degrees C. again. The lyophilizer chamber pressure was reduced to 10 mTorr and the shelf temperature was held constant at −40 degrees C. for 10 hours. The shelf temperature was then increased to 25 degrees C. for 10 hours. The resulting product exhibited excellent reconstitution properties as a result in inclusion of the annealing step, which resulted in recrystallization of the mannitol and a crystalline frozen structure.

In addition to fast reconstitution and maintenance of active reagents, it is usually also important for the pellets to have sufficient mechanical strength (i.e., not excessively friable) to withstand handling without breakage. This is important, in large part, because the intact pellet is sized to provide a defined quantity of reagent, and breakage, with associated reagent loss, results in an undetermined quantity of reagent remaining. This will typically result in undesirably erratic assay results. This requirement for pellet strength and resulting pellet integrity is particularly important for pellets which are individually dispensed for an individual assay as compared to assay for which very small pellets are dispensed by weight.

For pellets larger than about 2 mm up to about 5 mm, the pellets desirably have a compression strength of 2 to 25 grams as measured using a Chatillon TCD Series 110 Force Testing System (Willrich Precision Instruments TDD110-250g) or device giving substantially equivalent measurement, or a numerical measure determined using a different device which corresponds to the strength must indicated. In certain cases, the strength is desirably 4 to 10 grams, or 4 to 15 grams, or 7 to 15 grams, or 10 to 15, grams or 10 to 20 grams, or 15 to 25 grams, or about 8, 10, 12, or 15 grams. An alternative force testing system is the FTS-MG Force Testing System available, for example, through Albuquerque Industrial, Briarwood, N.Y.

For pellets in the size range of 0.25 mm to 2 mm, pellets desirably have a strength as specified for pellets larger than 2 mm, or advantageously in a range of 2 to 20 grams, 2 to 15 grams, 2 to 10 grams, 2 to 8 grams, 4 to 20 grams, 4 to 15 grams, 4 to 12 grams, 4 to 10 grams, 4 to 8 grams, 6 to 15 grams, 6 to 12 grams, or 6 to 10 grams.

For pellets in the size range of 0.1 mm to 0.25 mm the pellets desirably have a strength of 0.5 to 10 grams, 0.5 to 8 grams, 0.5 to 5 grams, 0.5 to 4 grams, 0.5 to 3 grams, 1 to 8 grams, 1 to 5 grams, 1 to 4 grams, 1 to 3 grams, 2 to 10 grams, 2 to 8 grams, 2 to 6 grams, 2 to 4 grams, 4 to 10 grams, 4 to 8 grams, or 4 to 6 grams.

As discussed above, a process for making fast reconstituting pellets with good activity can be accomplished in steps, with the process being especially applicable to protein-containing reagent solutions. Each protein or enzyme will, of course, require different conditions to optimize stability and reconstitution. For example, lyophilization of the enzyme lactate dehydrogenase in the absence of cryoprotectants or stablizers induces significant conformational changes and subsequent reductions in enzyme activity. The addition of any of 10 mM mannitol, 100 mM mannitol, 10 mM sucrose or 100 mM sucrose to the enzyme solution attenuates the unfolding (as can be shown with infrared spectroscopy) after lyophilization, but not completely. The result is a reduction in the enzyme activity. However, when a combination of 10 mM mannitol and 10 mM sucrose is added, the native structure is preserved during lyophilization and substantially full enzymatic activity is recovered after reconstitution.

Thus, the first step is to identify conditions which allow freezing and lyophilization while retaining activity upon reconstitution. This involves testing and identifying a set of cryoprotectants/stabilizers and optimum concentrations. Common cryoprotectants (which can also act as protectants during lyophilization) include various mono- and disaccharides, such as trehalose, mannitol, sorbitol, and sucrose, although others have also been used. Trehalose, in particular, has been found advantageous, commonly at concentrations of about 1 to 20%, and more often at about 4 to 15%, or 5 to 12% by weight. Generally, sugars such as those listed above, as well as others, can be used as protectants in concentrations ranging from about 0.1% to 20% or greater, although concentrations above about 20% can appreciably degrade reconstitution properties.

Once an appropriate reagent solution(s) is identified which provides acceptable cryo- and Iyo-protection, an optimum or at least acceptable lyophilization cycle is developed. For the present fast reconstituting pellets, it has been found in many cases that an annealing or thermal treatment step, is important to having a final product which exhibits rapid and complete reconstitution.

In general, ice structure and mass transfer resistance during primary drying can be altered by freezing the beads, loading the beads on a pre-cooled lyophilizer shelf (at least 10° C. below the glass transition temperature) and warming the frozen beads to a temperature just above the glass transition temperature. This allows the ice crystals to rearrange and grow to a more stable state. The frozen beads are then cooled again to a temperature at least 10° C. below the glass transition temperature and the primary lyophilization cycle is initiated. The lyophilized beads produced after annealing tend to have larger pores than those from the standard freezing method, which facilitate water-vapor transmission and lower the mass transfer resistance during primary drying, as well as improve reconstitution properties.

For example, in one test an annealing step was not utilized and the frozen product was initially placed on a pre-cooled −40 degree C. lyophilizer shelf. The lyophilizer chamber pressure was reduced to 10 mTorr and the shelf temperature was held constant at −40 degrees C. for 10 hours. The shelf temperature was then increased to 25 degrees C. for 10 hours. The resulting lyophilized product exhibited very poor reconstitution. In contrast, in a second test, a second lyophilization cycle was created that contained an annealing step. During this cycle the frozen product was initially placed on a pre-cooled −40 degree C. lyophilizer shelf. The chamber pressure was reduced slightly to 600 mTorr and the shelf temperature was increased to −30 degrees C., held at that temperature for 2 hours and reduced to −40 degrees C. again. The lyophilizer chamber pressure was reduced to 10 mTorr and the shelf temperature was held constant at −40 degrees C. for 10 hours. The shelf temperature was then increased to 25 degrees C. for 10 hours. The resulting product exhibited superior reconstitution as compared to the product in the previous test. This improvement was due to the addition of annealing step, resulting in recrystallization of the mannitol and a crystalline frozen structure.

In general, increasing concentration of sugars (frequently mannitol and/or sucrose) increases the hardness of the resulting pellets, but slows the reconstitution rate. As a result, identifying suitable conditions for creating fast reconstituting pellets can involve balancing pellet strength and reconstitution time by controlling the types and concentrations of sugars in the reagent solution, which balance also should not excessively reduce reagent activity in the reconstituted solutions. In many cases of the present pellets, a concentration of trehalose is used (e.g., at about 6 to 12%) primarily as a cryo- and lyo-protectant, and mannitol and/or sucrose (e.g., at about 1 to 2.5%) is added as additional protectant and to contribute to pellet strength. When frozen on the surface of liquid nitrogen and lyophilized using a thermal treatment step (e.g., as described herein for exemplary preparations), fast reconstituting pellets with excellent reconstituted reagent solution activity (particularly including thromboplastin activity) are obtained.

### B. Heterogeneous Pellets

The invention additionally provides heterogeneous pellets, i.e., pellets prepared from separate droplets of at least two different reagent solutions. The present heterogeneous pellets will, in many cases, be rapidly reconstituting pellets, but in some cases may be prepared such that they reconstitute more slowly or after a time delay. Generally heterogeneous pellets are prepared such that reagent components in the different reagent solutions do not substantially react with one another. Such heterogeneous pellets may be prepared in at least two different ways, resulting respectively in mixed heterogeneous pellets and un-mixed heterogeneous pellets.

It should be recognized multiple components may be mixed in a single reagent when the components do not react together to a significant degree or if it is desired for two or more of the components to react prior to their use in an assay or other process. Thus, the present heterogeneous pellets are primarily advantageous in cases where reaction between two or more components should not occur to a significant extent prior to resuspension.

Preventing reaction can be accomplished by having reagents components which would otherwise react in separate volumes of the pellet, or by allowing mixing but at sufficiently low temperature and followed by rapid freezing to prevent significant reaction from occurring.

For example, if there is a sufficient time interval between introductions of the separate droplets, the first droplet can begin freezing before the second droplet contacts the first. The result is that the two droplets will adhere, and, upon freezing, form a heterogeneous pellet such that the two solutions substantially occupy separate but adjacent spaces or volumes. Depending on the length of the time interval between droplet introductions, the physical form of the heterogeneous pellet can range from discrete, adhered pellets to coating of the first pellet with the second reagent solution. One or more additional reagent solutions can be introduced in droplet form in the same manner to form heterogeneous pellets containing three or more different reagent solutions.

Alternatively, separate droplets of two different solutions can be introduced into a freezing environment (e.g., the surface of liquid nitrogen). If the separate droplets are introduced essentially simultaneously (or sufficiently close in time that appreciable freezing of the first droplet has not occurred) such that the liquid drops combine, mixing of the two solutions can occur to at least some extent, but if the temperature is sufficiently low and/or freezing occurs sufficiently rapidly, the reaction(s) will be slowed and/or limited sufficiently that the limited extent of reaction can be acceptable.

This general approach is readily applied to relatively large droplets, e.g., droplets resulting in heterogeneous pellets above about 0.25 mm, but can also be applied to smaller droplets. In this approach, multi-solution droplets may be formed by merging of individually emitted single solution droplets. That is, co-targeted and timed droplets are directed so that they contact each other (but not with sufficient force to cause droplet splitting) above or on the surface of a cryogenic liquid. If droplets are to contact each other before they reach the surface of the cryogenic liquid, the timing of release of the respective droplets must be precisely coordinated. If the droplets are to contact each other on the surface of the cryogenic liquid the respective releases should still be substantially at the same time, but a small discrepancy can be tolerated. For very small droplets, the droplet release can, for example, be made using piezo nozzles, similar to piezo inkjet printer nozzles.

As yet another alternative to making pellets which contain mixed but non-reacted components from two or more separate reagent solutions (or as an additional technique in the production of mixed heterogeneous pellets as described above), the reagent solutions can be pre-cooled to temperatures (e.g., often about 1 to 4 degrees C.) such that when the solutions are combined, any reaction that occurs is limited to an acceptable level. The pre-cooled separate solutions may be combined, for example, just prior to forming the reagent solution droplet, producing a pre-cooled mixed solution droplet, which is rapidly frozen, for example, on the surface of a cryogenic liquid such as liquid nitrogen.

Hybrid reagent compositions with different characteristics have been prepared. For example, Roy, U.S. Pat. No. 4,712,310 (which is incorporated herein by reference in its entirety), describes the preparation of powders of hybrid lyophilized spray droplets. The hybrid spray droplets are formed by spraying two solutions containing incompatible components onto the same surface area of a cryogenic liquid such that hybrid droplets form, apparently with little mixing of solutions. After lyophilization the powders can be formed into tablets for use or can be used as dry powders. A limitation of this approach is the droplets are very small but cover a significant size range and therefore contain significantly varying amounts of reagent, and also there is a significant probability that a hybrid droplet will be formed from unequal numbers of droplets of the separate solutions. Thus, when closely controlled amounts of reagent are desired, sufficient numbers of powder particles must be used such that the interparticle variability becomes insignificant and the total amount of each reagent is governed by the average amount in larger numbers of particles. In the present invention, amounts of reagents are controlled in each pellet by forming heterogeneous pellets from controlled volumes of each solution. This process results in particles in which the quantity of each reagent is defined down to single pellets. The Roy patent describes types of incompatible components which may be included in the separate solutions; such incompatible components may be utilized in the present heterogeneous pellets (i.e., as the reacting components).

In making heterogeneous pellets, a person can make pellets of varying physical forms and test them for performance in the particular assay or other process of interest, and select pellets with particularly desirable performance characteristics.

As an example of heterogeneous pellets useful in thromboelastometry and similar assays, a combination of an activator (e.g., thromboplastin or other activator) and calcium can be beneficially made. Such a combination, can, for example, reduce the amount of filler otherwise utilized in pellets.

While heterogeneous pellets can be used to provide combination of reagents which will interact in solution, multiple components can also be provided in the form of separate pellets. This is particularly advantageous when very small pellets are used, e.g., in an assay. With very small pellets, quantities of each dried reagent solution can be measured out by weight rather than by single pellets or counting out small numbers of pellets. In addition, using separate very small pellets in this manner provides the separate reagents in close proximity so that upon reconstitution the separate reagents can interact very quickly.

### C. Pellets for Thromboelastometry and Other Hemostasis Assays

It was found that fast reconstituting pellets could advantageously be produced and used in thromboelastometry and other hemostasis assays. In certain of these assays, it is important for the reagents to be present and active nearly immediately upon contact of the test sample and the reagent mixture. As a result, the standard in these types of assays has been to use liquid reagents, though attempts have been made to use dry reagents. However, thromboelastometry assay tests performed using dry reagents prepared from solution containing tissue factor substantially according to Calatzis et al., US Pat Appl Publ 2010/0190193 (which is incorporated herein by reference in its entirety) resulted in highly variable assay results, with the variations of sufficient magnitude to render assay result interpretation very difficult or even impossible.

Despite those difficulties, it was discovered that fast reconstituting pelletized reagent compositions containing the same active components as used in the just described unsuccessful tests gave quite reproducible results, even without process and composition optimization.

Thus, it was determined that very useful fast reconstituting pellets are provided which contain tissue factor (thromboplastin) or other protein component of a thromboelastometry or other hemostasis assay. Examples of components which may be incorporated in the present reagent pellets are indicated in the Calatzis patent application cited above.

### D. Pellet Preparation Examples

Below are described the preparations of three different size ranges of pellets, including specific examples of pellets in each size range.

1. Beads Greater than about 2 mm Diameter (Greater than 5 uL)

Formulation:

Typically the formulation of a liquid reagent being used in lyophilization is very important in determining the performance and physical characteristics of the resulting lyophilized material. For the present pellets, the formulation is selected such that the resulting lyophilized product exhibits the characteristics deemed desirable, including room-temperature stability, friability resistance, and fast reconstitution times. Usually the formulation will employ sugars to stabilize proteins, stabilizers against specific degradation mechanisms such as an anti-oxidant, bulking agents, buffering agents, as well as other excipients.

Dispense Method:

Those who have knowledge in liquid dispensing will recognize that most conventional methods of accurately dispensing liquid reagents can be used to form a drop with a volume of greater than about 5 uL or more with an accuracy of approximately +/−1% or better. Such liquid dispensing methods can include pipettes, syringe pumps, metering pumps, and other positive displacement pumps.

Method of Freezing:

During this stage of the lyophilization process, the formulation is cooled. Pure crystalline ice forms from the liquid, thereby resulting in a freeze concentration of the remainder of the liquid to a more viscous state that inhibits further crystallization. The highly concentrated and viscous solution solidifies, yielding an amorphous, crystalline, or combined amorphous-crystalline phase.

The drops are added individually to a cryogenic liquid, such as liquid nitrogen, which is approximately −196 degrees C. During the time when the drop is freezing, it is desirable to isolate that drop. This can be accomplished by utilizing an aluminum block with a series of through holes. A block with through holes each having a diameter of approximately 0.75 inches can be effectively used, although other size holes (including smaller holes) can also be used. At the bottom of the vessel containing the cryogenic liquid conveniently rests a tray, typically fabricated utilizing black anodized aluminum. The drop falls to the bottom of the vessel after freezing and is collected on the aluminum tray.

Method of Freezing (Heterogeneous Beads):

Under some circumstances, two or more liquid components may be added to a single bead. However, in many cases the components should not substantially mix with each other in their liquid states due to the possibility of degradation or loss of activity of one or more of the liquid components or premature production of reaction product. In such cases, the drops of two or more liquid components may be added to a single compartment or hole in the aluminum block. A drop of each component is dispensed in a sequential fashion, thereby allowing the preceding drop to decrease in temperature to minimize interaction with the next component drop. In most cases for pellets of this size range, there is a dwell time of between fractions of one second and 10 seconds between the dispensing of each of the liquid components.

Transfer of Frozen Beads to the Lyophilizer:

The tray containing the frozen beads is then transferred to a pre-cooled lyophilizer shelf. The shelf temperature is usually lower than the melting point of the frozen beads, commonly −40 degrees C.

Lyophilization Cycle:

A series of experiments can be carried out to determine an effective or optimum lyophilization cycle. Typically the lyophilization cycle consists of a thermal treatment, a primary dry and a secondary dry. The thermal treatment is used to allow the frozen product to recrystallize, resulting in a more stabile and better performing product. As an example, thermal treatment may consist of an increase of shelf temperature from −40 degrees C. to −30 degrees C. (e.g., over 30 minutes), the shelf temperature is then held at −30 degrees (e.g., for 60 minutes), and the shelf is recooled to −40 degrees C.

During the primary dry phase, ice formed during freezing is removed by sublimation at sub-ambient temperatures under vacuum. This step is usually is carried out at chamber pressures of 40-400 Torr and shelf temperatures ranging from −50 degrees C. to 10 degrees C. (commonly about −40 degrees C.). Throughout this stage, the product is maintained in the solid state below the collapse temperature of the product in order to dry the product with retention of the structure established in the freezing step. The collapse temperature is the glass transition temperature (Tg) in the case of amorphous products or the eutectic temperature (Te) for crystalline products. As an example, the pressure can be reduced from one atmosphere to 40 Torr, and the shelf temperature will increase from −40 degrees C. to 0 degrees C. over a 12 hour period.

The final portion of the lyophilization cycle is the second dry phase. Some or substantially all of the relatively small amount of bound water remaining in the matrix is removed by desorption. During this stage, the temperature of the shelf and product are increased to promote adequate desorption rates and achieve the desired residual moisture. Typically, the shelf temperature is increased to 25 degrees C. and the chamber pressure remains at 40 microns, e.g., for 4 hours or more. The degree of drying can be adjusted to maximize stability and activity of the reagents.

Product Packaging:

Typically the product is removed from the lyophilizer in a room or area that has very low moisture, typically about 5% relative humidity (RH). The beads are packaged in hermetic containers that usually contain desiccant. The beads can later be placed in individual packages by automated equipment.

Quality Control:

The beads are usually tested to make certain they meet or exceed specifications. The specifications usually include hardness, residual moisture, and reconstitution time, for example, as described for the following example.

### Example 1

### Production of 2 mm Antibody Pellets

Formulation:

50 mg/ml hCG mouse monoclonal antibody (abcam ab400)

### 15% Trehalose (Sigma T9531)

### 1.8% Mannitol (Sigma M9546)

Dispensing and Freezing Method:

An appropriate container is filled with liquid nitrogen. At the bottom of the container is a lyophilization tray. An aluminum block with 0.75 inch diameter through holes spaced 1.25 inch center-to-center is lowered into the liquid nitrogen. A Gilson Pipetman P20 (Gilson F123600) with appropriate Gilson disposable tips (Gilson F171200) is used to dispense a 20 uL drop into the center of one of the holes in the aluminum block. Another drop is dispensed into the next through hole, etc., allowing the previously dispensed drops to freeze and fall to the bottom of the container where they are collected by the tray. This dispensing step is continued until the desired number of drops are dispensed, forming the desired number of frozen pellets.

Lyophilization:

The lyophilization tray containing the frozen beads is moved to a pre-cooled, −40 degrees C. shelf of a VirTis Advantate 2.0 EL lyophilizer. The following, pre-programmed cycle is initiated (all time is in minutes and temperatures are in degrees C.).

| Temperature | Time | Ramp/Hold |
|---|---|---|
| Thermal Treatment | | |
| Phase | | |
| −40 | 30 | Hold |
| −30 | 60 | Ramp |
| −40 | 60 | Ramp |
| −40 | 30 | Hold |
| Primary Dry Phase | | |
| −40 | 600 | Hold |
| −20 | 120 | Ramp |
| 0 | 120 | Ramp |
| Secondary Dry | | |
| Phase | | |
| 25 | 120 | Ramp |
| 25 | 120 | Hold |

Product Packaging:

At the completion of the lyophilization cycle, a custom glove box which surrounds the lyophilizer door is filled with dry nitrogen gas. An air bleed cycle is initiated in which dry nitrogen gas is injected into the lypohilization chamber until the chamber is at ambient pressure. The operator opens the lyophilizer door and transfers the lyophilized beads to a Thermo Scientific Nunc 50 mL conical-bottom disposable plastic tube (Cole Parmer EW-17411-10) containing a custom screen. Beneath the screen is Drierite Indicating desiccant, 8 mesh (Drierite 23005).

Quality Control:

The moisture content of a bead was determined utilizing an AquaStar Karl Fischer Titrator AQV21 (EMD Chemicals AXAQV21). The average moisture content of a bead was approximately 1%. The hardness of the bead was tested using a Chatillon TCD Series 110 Force Testing System (Willrich Precision Instruments TDD110-250g) and was found to be approximately 12 grams. The reconstitution time is a qualitative visual test and was found to be less than one second and acceptable.

2. Beads Greater than about 0.25 mm (Greater than 0.1 uL) and Less than about 2 mm Diameter (Less than 5 uL)

Formulation:

As with the larger pellets described above, typically the formulation of a liquid reagent being used in lyophilization is important in determining the characteristics of a lyophilized product. Thus, a formulation is selected which will result in lyophilized product exhibiting the selected desirable characteristics, including room-temperature stability, friability resistance, and fast reconstitution times. Usually the formulation will employ sugars to stabilize proteins, stabilizers against specific degradation mechanisms such as an anti-oxidant, bulking agents, buffering agents, as well as other excipients. An exemplary formulation may contain about 15% trehalose and 7.2% mannitol.

Dispense Method:

Persons who have knowledge in liquid dispensing will recognize that conventional methods of accurately dispensing liquid reagents can be used to form a drop within the volume range of about 0.1 microliter to 5 microliter with an accuracy of about +/−1%. Such liquid dispensing methods can include linear ceramic pumps and other precision pumps. The most important aspect of this type of pump is to form individual drops that are expelled from the tip of the dispenser.

Method of Freezing:

During this stage the formulation is cooled. Pure crystalline ice forms from the liquid, thereby resulting in a freeze concentration of the remainder of the liquid to a more viscous state that inhibits further crystallization. The highly concentrated and viscous solution solidifies, yielding an amorphous, crystalline, or combined amorphous-crystalline phase.

The drops are added individually to a cryogenic liquid, such as liquid nitrogen, which is approximately −196 degrees C. During the time when the drop is freezing, it is desirable to isolate that drop. As with the larger pellets discussed above, this can be accomplished by utilizing an aluminum block with a series of through holes. While the diameter of the through holes can vary, holes with diameters of approximately 0.25 inches are effective for this range of droplet volumes (and resulting pellet sizes). For convenient collection of frozen droplets, a tray, typically fabricated utilizing black anodized aluminum, can rest at the bottom of the vessel containing the cryogenic liquid. The drop falls to the bottom of the vessel after freezing and is collected on the aluminum tray.

Method of Freezing (Heterogeneous Beads):

Under some circumstances, two or more liquid components may be added to a single bead. However, the components should substantially not mix with each other in their liquid states due to the possibility of degradation or loss of activity of one or more of the liquid components. In such cases, the drops of two or more liquid components may be added to a single compartment or hole in the aluminum block. A drop of each component is dispensed in a sequential fashion, thereby allowing the preceding drop to decrease in temperature to minimize interaction with the next component drop. For drops in this size range, there is usually a dwell time of between fractions of one second and 5 seconds between the dispensing of each of the liquid components.

Transfer of Frozen Beads to the Lyophilizer:

The tray containing the frozen beads is then transferred to a pre-cooled lyophilizer shelf. The shelf temperature is usually cooler than the melting point of the frozen beads, typically −40 degrees C.

Lyophilization Cycle:

A series of experiments can be completed to determine an effective or optimum lyophilization cycle. Typically the lyophilization cycle consists of a thermal treatment, a primary dry and secondary dry. The thermal cycle is used to allow the frozen product to recrystallize, resulting in a more stabile and better performing product. As an example, thermal treatment may consist of an increase of shelf temperature from −40 degrees C. to −30 degrees C. over 30 minutes, the shelf temperature is then held at −30 degrees for 60 minutes, and the shelf if cooled to −40 degrees C. again.

During the primary dry phase, ice formed during freezing is removed by sublimation at sub-ambient temperatures under vacuum. This step traditionally is carried out at chamber pressures of 40-400 Torr and shelf temperatures ranging from −50 degrees C. to 10 degrees C. Throughout this stage, the product is maintained in the solid state below the collapse temperature of the product in order to dry the product with retention of the structure established in the freezing step. The collapse temperature is the glass transition temperature (Tg) in the case of amorphous products or the eutectic temperature (Te) for crystalline products. The primary dry is typically the portion of the lyophilization cycle where the majority of the moisture is removed. As an example, the pressure will be reduced from one atmosphere to 40 microns, and the shelf temperature will increase from −40 degrees C. to 0 degrees C. over a 12 hour period.

The final portion of the lyophilization cycle is the second dry phase. Some or substantially all of the relatively small amount of bound water remaining in the matrix is removed by desorption. During this stage, the temperature of the shelf and product are increased to promote adequate desorption rates and achieve the desired residual moisture. Typically, the shelf temperature is increased to 25 degrees C. and the chamber pressure remains at 40 microns for 4 hours.

Product Packaging:

Typically the product is removed from the lyophilizer in a room or area that has very low moisture, typically about 5% RH. The beads are packaged in hermetic containers that usually contain desiccant. The beads can later be placed in individual packages by automated equipment.

Quality Control:

The beads are usually tested to make certain they meet or exceed specifications. The specifications usually include hardness, residual moisture, and reconstitution time, for example as described for the example below.

### Example 2

### Preparation of about 1.5 mm Heterogeneous Pellets

Test Formulation:

### Solution A:

0.05M Acetic acid (Sigma 34254)
0.004% Bromothymol Blue solution (Sigma 318752)

### 4% Mannitol (Sigma M9546)

### Solution B:

### 0.1M Sodium Bicarbonate (Sigma 36486)

### 4% Mannitol (Sigma M9546)

Dispensing and Freezing Method:

An appropriate container is filled with liquid nitrogen. At the bottom of the container is a lyophilization tray. An aluminum block with 0.50 inch diameter through holes spaced 1.25 inch center-to-center is lowered into the liquid nitrogen. An IVEK Digispense 3009 with a 3 A Cermic Pump module is used to dispense a 2 uL drop of Solution A into the center of one of the holes in the aluminum block. Approximately two seconds later a 2 uL drop of Solution B is dispensed with a second IVEK pump into the center of the same hole in the aluminum block. The Solution A drop is sufficiently frozen so that there is not an appreciable interaction with Solution B. If a reaction had taken place, it would be indicated by the development of a blue color because the bromothymol blue in Solution A, normally a green color in an acidic solution, would change to blue in a basic solution. The Solution B drop covers and combines with the sufficiently frozen A drop. The dispensing of Solution A and Solution B is continued in a similar manner in the next through hole, allowing the previously dispensed drops to freeze and fall to the bottom of the container where it is collected by the tray. This dispensing method is continued until the desired number of drops is dispensed.

Lyophilization:

The lyophilization tray containing the frozen beads is moved to a pre-cooled, −40 degrees C. shelf of a VirTis Advantate 2.0 EL lyophilizer. The following, pre-programmed cycle is initiated (all time is in minutes and temperatures are in degrees C.).

| Temperature | Time | Ramp/Hold |
|---|---|---|
| Thermal Treatment | | |
| Phase | | |
| −40 | 30 | Hold |
| −25 | 60 | Ramp |
| −40 | 60 | Ramp |
| −40 | 30 | Hold |
| Primary Dry Phase | | |
| −40 | 400 | Hold |
| −20 | 60 | Ramp |
| 0 | 60 | Ramp |
| Secondary Dry Phase | | |
| 25 | 60 | Ramp |
| 25 | 120 | Hold |

Product Packaging:

At the completion of the lyophilization cycle, a custom glove box which surrounds the lyophilizer door is filled with dry nitrogen gas. An air bleed cycle is initiated in which dry nitrogen gas is injected into the lypohilization chamber until the chamber is at ambient pressure. The operator opens the lyophilizer door and transfers the lyophilized beads to a Thermo Scientific Nunc 50 mL conical-bottom disposable plastic tube (Cole Parmer EW-17411-10) containing a custom screen. Beneath said screen is Drierite Indicating desiccant, 8 mesh (Drierite 23005).

Quality Control:

The moisture content of a bead was determined utilizing an AquaStar Karl Fischer Titrator AQV21 (EMD Chemicals AXAQV21). The average moisture content of a bead was approximately 0.75%. The hardness of the bead was tested using a Chatillon TCD Series 110 Force Testing System (Willrich Precision Instruments TDD110-250g) and was found to be approximately 8 grams. The reconstitution time is a visual qualitative test and was found to be less than one second and acceptable.

3. Beads Greater than 10 um (Greater than 1 pL) and Less than 0.25 mm (Less than 0.1 uL)

Formulation:

Typically the formulation of a liquid reagent that will be used in lyophilization is critical. The resulting lyophilized product will exhibit the characteristics deemed desirable, including room-temperature stability, friability resistance, and fast reconstitution times. Usually the formulation will employ sugars to stabilize proteins, stabilizers against specific degradation mechanisms such as an anti-oxidant, bulking agents, buffering agents, as well as other excipients. A typical formulation may contain about 15% trehalose and 7.2% mannitol.

Dispense Method:

Most who have knowledge in liquid dispensing would appreciate that conventional methods of accurately dispensing liquid reagents can be used to form a drop in the volume range of about 1 pL to 1 microliter with an accuracy of about +/−5%. Such liquid dispensing methods can include piezoelectric nozzles.

Method of Freezing:

During this stage the formulation is cooled. Pure crystalline ice forms from the liquid, thereby resulting in a freeze concentration of the remainder of the liquid to a more viscous state that inhibits further crystallization. The highly concentrated and viscous solution solidifies, yielding an amorphous, crystalline, or combined amorphous-crystalline phase.

The drops are added individually to a cryogenic liquid, such as liquid nitrogen, which is approximately −196 degrees C. During the time when the drop is freezing, it is desirable to isolate that drop. This can be accomplished by utilizing an aluminum block with a series of through holes, each with a diameter of approximately 0.10 inches. At the bottom of the vessel containing the cryogenic liquid rests a tray, typically fabricated utilizing black anodized aluminum. The drop falls to the bottom of the vessel after freezing and is collected on the aluminum tray.

Method of Freezing (Heterogeneous Beads):

Under some circumstances, two or more liquid components may be added to a single bead. However, the components should not mix with each other in their liquid states due to the possibility of degradation or loss of activity of one or more of the liquid components. In such cases, the drops of two or more liquid components may be added to a single compartment or hole in the aluminum block. A drop of each component is dispensed in a sequential fashion, thereby allowing the preceding drop to decrease in temperature to minimize interaction with the next component drop. There is a commonly a dwell time of between fractions of one second and 5 seconds between the dispensing of each of the liquid components. Usually a shorter dwell time is used with the smaller droplets.

Transfer of Frozen Beads to the Lyophilizer:

The tray containing the frozen beads is then transferred to a pre-cooled lyophilizer shelf. The shelf temperature is usually cooler than the melting point of the frozen beads, typically −40 degrees C.

Lyophilization Cycle:

A series of experiments is completed to determine the optimum lyophilization cycle. Typically the lyophilization cycle consists of a thermal treatment, a primary dry and secondary dry. The thermal cycle is used to allow the frozen product to recrystallize, resulting in a more stabile and better performing product. As an example, thermal treatment may consist of an increase of shelf temperature from −40 degrees C. to −30 degrees C. over 30 minutes, the shelf temperature is then held at −30 degrees for 60 minutes, and the shelf if cooled to −40 degrees C. again.

During the primary dry phase, ice formed during freezing is removed by sublimation at subambient temperatures under vacuum. This step traditionally is carried out at chamber pressures of 40-400 Torr and shelf temperatures ranging from −50 degrees C. to 10 degrees C. Through out this stage, the product is maintained in the solid state below the collapse temperature of the product in order to dry the product with retention of the structure established in the freezing step. The collapse temperature is the glass transition temperature (Tg) in the case of amorphous products or the eutectic temperature (Te) for crystalline products. The primary dry is typically the portion of the lyophilization cycle where the majority of the moisture is removed. As an example, the pressure will be reduced from one atmosphere to 40 microns, and the shelf temperature will increase from −40 degrees C. to 0 degrees C. over a 12 hour period.

The final portion of the lyophilization cycle is the second dry phase. The relatively small amount of bound water remaining in the matrix is removed by desorption. During this stage, the temperature of the shelf and product are increased to promote adequate desorption rates and achieve the desired residual moisture. Typically, the shelf temperature is increased to 25 degrees C. and the chamber pressure remains at 40 microns for 4 hours.

Product Packaging:

Typically the product is removed from the lyophilizer in a room or area that has very low moisture, typically about 5% RH. The beads are packaged in hermetic containers that usually contain desiccant. The beads can later be placed in individual packages by automated equipment.

Quality Control:

The beads are usually tested to make certain they meet or exceed specifications. The specifications usually include hardness, residual moisture, and reconstitution time.

### Example 3

### Production of Test Heterogeneous Pellets of about 0.25 mm

Formulation:

### Solution A:

### 1M Citric Acid Monohydrate (Sigma C1909)

### 4% Mannitol (Sigma M9546)

### Solution B:

### 1M Sodium Bicarbonate (Sigma S5761)

### 4%. Mannitol (Sigma M9546)

Dispensing and Freezing Method:

An appropriate container is filled with liquid nitrogen. At the bottom of the container is a lyophilization tray. An aluminum block with 0.125 inch diameter through holes spaced 0.5 inch center-to-center is lowered into the liquid nitrogen. A modified Nandrop I dispensing system (Innovadyne Technologies, Inc.) is used to dispense a 25 nL drop of Solution A into the center of one of the holes in the aluminum block. Approximately 0.5 seconds later a 75 nL drop of Solution B is dispensed with a second modified Nanodrop I dispensing system into the center of the same hole in the aluminum block. The Solution A drop is sufficiently frozen so that there is not substantial interaction with Solution B. The Solution B drop covers and combines with the sufficiently frozen A drop. The dispensing of Solution A and Solution B is continued in a similar manner in the next through hole, allowing the previously dispensed drops of Solution A and Solution B to freeze and fall to the bottom of the container where it is collected by the tray. This dispensing method is continued until the desired number of drops is produced.

Note that when Solution A and Solution B are combined in their liquid form, carbon dioxide gas is generated. The reaction would take place as shown below.

H₃C₆H₅O₇.H₂O+3NaHCO₃→Na₃C₆H₅O₇+4H₂O+3CO₂

According to the reaction above, it is desirable to combine Solution A and Solution B in a 1:3 ratio, as is indicated by the dispensing of volume of Solution A and Solution B. This example would indicate if a reaction had taken place in the solution form as carbon dioxide would be liberated.

Lyophilization:

The lyophilization tray containing the frozen beads is moved to a pre-cooled, −40 degrees C. shelf of a VirTis Advantate 2.0 EL lyophilizer. The following, pre-programmed cycle is initiated (all time is in minutes and temperatures are in degrees C.).

| Temperature | Time | Ramp/Hold |
|---|---|---|
| Thermal Treatment | | |
| Phase | | |
| −40 | 30 | Hold |
| −30 | 30 | Ramp |
| −40 | 15 | Ramp |
| −40 | 15 | Hold |
| Primary Dry Phase | | |
| −40 | 120 | Hold |
| −20 | 120 | Ramp |
| 0 | 120 | Ramp |
| Secondary Dry | | |
| Phase | | |
| 25 | 60 | Ramp |
| 25 | 60 | Hold |

Product Packaging:

At the completion of the lyophilization cycle, a custom glove box which surrounds the lyophilizer door is filled with dry nitrogen gas. An air bleed cycle is initiated in which dry nitrogen gas is injected into the lypohilization chamber until the chamber is at ambient pressure. The operator opens the lyophilizer door and transfers the lyophilized beads to a Thermo Scientific Nunc 50 mL conical-bottom disposable plastic tube (Cole Parmer EW-17411-10) containing a custom screen. Beneath said screen is Drierite Indicating desiccant, 8 mesh (Drierite 23005).

Quality Control:

The moisture content of 2 mg of the product was determined utilizing an AquaStar Karl Fischer Titrator AQV21 (EMD Chemicals AXAQV21). The average moisture content of a bead was approximately 0.75%. The hardness of the beads was not assessed due to the small size of the beads, although hardness determinations can be performed using equipment adapted to test small beads The reconstitution time is a visual qualitative test and was found to be less than one second and acceptable.

Dispenser Packaging

In numerous applications (e.g., for performing biological or biochemical assays), it is beneficial to minimize handling of the fast reconstituting (fast dissolving) reagent pellets by packaging them appropriately. In this way the pellets can be pre-distributed (e.g., in single-use format) ready for use in an assay or the like, so that the pellets do not need to be directly handled by the user. Furthermore multiple pellets may be packaged together, which may the same or different, e.g., pellets containing different reagents.

A highly advantageous approach is to package one or more of the pellets (single reagent and/or heterogeneous pellets) within a disposable pipette tip, the capacity of which is selected to be appropriate for the intended application. In most cases, the pipette tip will be sealed within a moisture-proof container, often a single-use container, for example, a sealed pouch, bubble pack, or blister pack. It may be desirable to include in the pipette tip a structure to retain the pellet(s) within the tip. In many cases, the pellet(s) will be sized such that they cannot pass through the dispensing orifice of the pipette tip. A pellet-retaining structure, e.g., a filter disc, which has perforations sufficiently small to retain the pellet(s) can be located within the pipette tip such that the pellet(s) is retained between the pipette tip dispensing orifice and the pellet-retaining structure. In some cases, such as when pellets smaller than the dispensing orifice of the pipette tip, a pellet retaining structure can also be included such that the pellets are retained by a pellet retaining structure between the pipette tip dispensing orifice and the pellets.

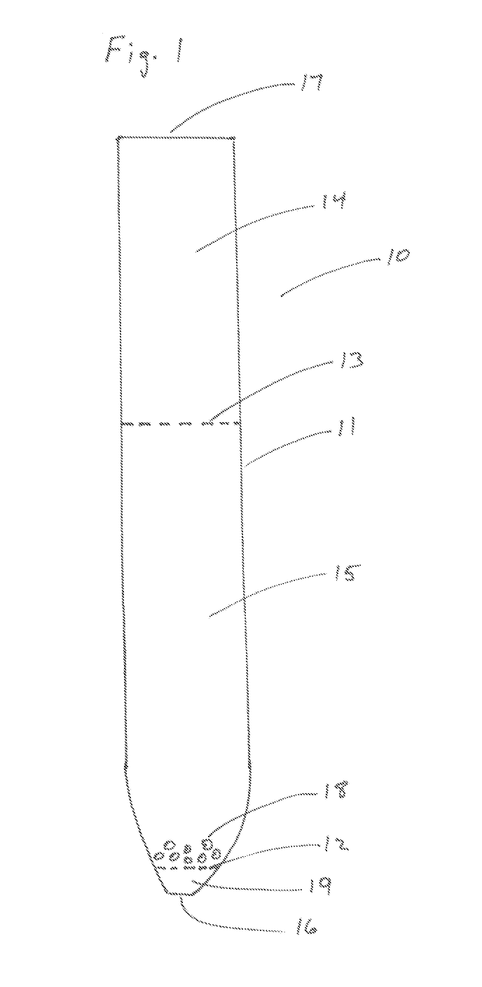

An illustration of such a pipette tip containing reagent pellets is provided in FIG. 1. As shown, the pipette tip 10 has a hollow body 11, with optional porous retention barrier 12, optional porous distal barrier 13, upper internal volume 14, lower internal volume 15, an inlet orifice 16, and a distal opening 17. The reagent pellets 18 are retained within the pipette tip because they are larger than the inlet orifice or are retained by optional retention barrier 12. When a retention barrier 12 is present, it is usually desirable to position the barrier in or as close as practical to the inlet orifice in order to minimize the volume of the dead space 19. If the porous retention barrier and/or the porous distal barrier are not present, the internal volumes 14, 15, and/or 19 will, or course, form a continuous volume or space due to the absence of the respective barrier(s).

In many cases the pellets will rapidly take up water vapor, e.g., from surrounding air. Therefore, it can also be beneficial to protect the pellets from water vapor such as by including a desiccant within the moisture-proof container or within the pipette tip itself. For example, desiccant can be held by the pellet-retaining structure, such as trapping desiccant granules between layers of filter material or other material having openings sufficiently small to retain the desiccant granules.

Any fast reconstituting reagent pellets may be used in the pipette tips as described, and particularly fast reconstituting reagent pellets as described herein.

Reagents to be used in the pellets packaged within disposable pipette tips can be of many different types. For example, the reagents can include proteins, nucleic acids, and inorganic salts, among others. The pellets can include buffer solution components, reaction enzymes and/or substrates, binding molecules, reaction activators or inhibitors, labels, and/or other types of reagent components, as well as suitable reagent combinations.

When a plurality of pellets is packaged within a disposable pipette tip, the pellets may be of one or multiple types. For example, pellets containing different reagents may be packaged together in a pipette tip, e.g., 2, 3, 4, 5, 6, or even more different reagent pellets. Of course, there may be one or a plurality of pellets of each particular reagent type. Pellets of different sizes may be included in a pipette tip. In some cases, it may be desirable to include pellets of different sizes but the same regent composition (e.g., to be able to use standardized pellets while adjusting the amount of the particular reagent in the tip), but in most cases, pellets of different size will be pellets of different reagent composition. A tip may contain different numbers of pellets of each different type of reagent composition.

In use, the pipette tips containing fast reconstituting reagent pellets are very advantageous in many applications because they minimize needed steps. The single step of taking up the liquid (e.g., sample, buffer solution, or water) into the pipette tip essentially immediately provides the needed volume of mixed reagent solution ready for dispensing into any suitable container. For example, in an assay in which it is desirable to mix reagents with a biological sample, drawing the defined volume of liquid sample into the pipette tip adds the appropriate amount of reagent to the sample, and also causes mixing of the reagent and liquid sample. Mixing is further enhanced by dispensing the sample/reagent mixture into a container.

To use the pellet-containing pipette tips, the tip placed on a suitable pipette (e.g., after opening a container enclosing the tip), and a volume of liquid appropriate for the particular assay or other intended application is drawn into the pipette tip. The pellet(s) dissolve rapidly, and the liquid with the dissolved reagent(s) is dispensed into a suitable receptacle.

In general, this approach provides both reagent dissolution and mixing, such that the concentration of the dissolved reagent(s) very quickly becomes essentially uniform throughout the liquid in the pipette tip. Additional mixing occurs during dispensing, further ensuring uniformity of reagent in the dispensed volume.

Such pipette tips containing rapidly reconstituting reagent pellets will often be packaged individually, e.g., in bubble packs or in sealed sleeves or the like. In many cases, the pipette tips will be sterile.

Sample Preparation Dispensing Tips

A particular application of reagent pellets in dispensing devices is for sample preparation, where a sample preparation process different from or in addition to mixing of sample and reagent occurs within the device. That is, a liquid biological (e.g., blood) or other sample is drawn into the device. If one or more reagent pellets are within the device, the pellet(s) dissolve in the sample. The sample preparation process occurs, and the prepared sample can be dispensed. The sample preparation process may, for example, be a separation of certain sample components, for example, blood cells. In some designs, the device is disposable, e.g., as a single-use device. Sample preparation may, for example, include addition of a sample preservative, addition of an anti-coagulant to a blood sample, removal of a component from a sample, such as removal of cells (e.g., blood cells), among others.

### DEFINITIONS

As used herein, the term “reagent pellet” or simply “pellet” refers to a dry reagent particle (e.g., assay reagent) prepared from a droplet of liquid reagent solution by drying the droplet (usually be lyophilization) without subsequent milling or other mechanical fracturing of the dry particle. In most cases, the droplet is solidified by freezing, and then dried using lyophilization. The term “pellet” is used herein synonymously with the term “bead”.

The term “heterogeneous pellet” as used herein refers to a dry reagent pellet formed from at least two different reagent solutions. A “mixed heterogeneous pellet” is a heterogeneous pellet in which at least two different reagent solutions are mixed prior to freezing and drying. An “un-mixed heterogeneous pellet” is a heterogeneous pellet in which the reagent solutions are not substantially mixed prior to freezing and drying.

The term “milli-pellet” is used herein to refer to pellets which have average cross-sectional dimensions of 0.5 mm or more. Similarly, “micro-pellet” is used herein to refer to pellets which have average cross-sectional dimension of less than 1 mm, often in the 1 to 400 micrometer range or 10 to 200 micrometer range.

Indication that the present pellets reconstitute (i.e., dissolve) within a specified time means the pellet dissolves within the specified time without forced fluid flow, e.g., due to pressurized fluid flow or fluid flow caused by centrifugation.

Indication that a device is “cylindrical” or “generally cylindrical” means that a significant portion of the device is approximately cylindrical, but one or more other portions may be non-cylindrical. For example, the bodies of most syringes include a cylindrical barrel, but also include an end section which tapers sharply to an inlet orifice. Similarly, many of the larger volume disposable pipette tips include a generally cylindrical portion (usually slightly tapering) but taper to a small inlet orifice.

As used herein in connection with sample preparation, the term “aggregation” means the clumping together of a particular component, e.g., blood cells. Aggregation thus includes agglutination. An “aggregating agent” or “aggregation agent” therefore refers to a chemical entity or combination of entities which will cause aggregation of the cells or other specified component. Examples of blood cell aggregating agents include suitable antibodies and lectins, among others.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

1-131. (canceled)

132. A dry, fast reconstituting heterogeneous reagent pellet, comprising
a first reagent solution; and
a second reagent solution,
wherein said first reagent solution and said second reagent solution respectively contain reagent components which will react together in mixed solution and are not substantially reacted in said pellet.

133. The dry, fast reconstituting heterogeneous reagent pellet of claim 132, wherein said pellet comprises at least two different reagent solution volumes.

134. The dry, fast reconstituting heterogeneous reagent pellet of claim 132, wherein said pellet comprises at least three different reagent solution volumes.

135. The dry, fast reconstituting heterogeneous reagent pellet of claim 132, wherein at least one of said reagent solutions comprises an enzyme.

136. The dry, fast reconstituting heterogeneous reagent pellet of claim 132, comprising at least one coagulation activator or other coagulation assay reagent.

137. The dry, fast reconstituting heterogeneous reagent pellet of claim 132, wherein said first reagent solution and said second reagent solution are mixed.

138. The dry, fast reconstituting heterogeneous reagent pellet of claim 137, wherein said pellet is lyophilized and said first reagent solution and said second reagent solution are mixed at a temperature sufficiently low that substantial reaction does not occur.

139. The dry, fast reconstituting heterogeneous reagent pellet of claim 132, wherein said first reagent solution is coated by said second reagent solution.

140. A method for preparing dry heterogeneous reagent pellets, comprising
placing a plurality of small droplets of a first reagent solution in a cryogenic environment; and
prior to full freezing of said first reagent solution, contacting said droplets of first reagent solution with droplets of a second reagent solution, thereby creating un-mixed heterogeneous pellets comprising a first reagent volume and an adhered second reagent volume without substantial mixing of said first and reagent solutions.

141. The method of claim 140, further comprising contacting reagent pellets containing said first and second reagent solutions with small droplets of a third reagent solution thereby forming three-reagent solution un-mixed heterogeneous pellets, wherein said third reagent solution does not substantially mix with said first or second reagent solution.

142. The method of claim 140, wherein said small droplets of said first reagent solution are isolated when contacted with said droplets of second reagent solution.

143. The method of claim 140, wherein at least one reagent component in said first reagent solution would substantially react with at least one component in said second regent solution when mixed at a temperature of 15 degrees C.

144. The method of claim 143, wherein said reagent component in said first reagent solution will specifically bind to said component in said second reagent solution.

145. A single use assay cartridge, comprising
a reagent container comprising a housing with a reagent well containing a single use quantity of a dry, pelletized reagent composition selected for use in an in vitro biological reaction, comprising reagent pellets which dissolve within 1 second or less in use for said biological reaction; and
a liquid sample deposition zone, wherein liquid sample deposited in said deposition zone reconstitutes said dry, pelletized reagent, and
wherein said assay cartridge provides an indication of the presence or amount of one or more parameters of said liquid sample.

146. The cartridge of claim 145, adapted for use of a blood, plasma, or serum sample.

147. The cartridge of claim 145, wherein said dry, pelletized reagent composition comprises a detectable label.

148. The cartridge of claim 145, wherein said dry pelletized reagent composition comprises an analyte binding agent.

149. The cartridge of claim 145, wherein said dry, pelletized reagent composition comprises an enzyme.

150. The cartridge of claim 145, wherein said in vitro biological reaction is a protein cleavage reaction.
